(19) United States
(12) Patent Application Publication
(10) Pub. No.: US 2023/0334998 A1
(43) Pub. Date: Oct. 19, 2023

(54) SURGICAL TEACHING AUXILIARY SYSTEM USING VIRTUAL REALITY AND METHOD THEREOF

(71) Applicant: SMART SURGERY, Taipei City (TW)

(72) Inventors: Yu-Chieh LEE, Taipei City (TW); Yi-Ta SHEN, Taipei City (TW); Hsin-Man CHIANG, Taipei City (TW)

(21) Appl. No.: 18/132,369

(22) Filed: Apr. 8, 2023

Related U.S. Application Data
(60) Provisional application No. 63/330,792, filed on Apr. 14, 2022.

(51) Int. Cl. G06F 3/01; G06T 17/00; G09B 5/02

(52) U.S. Cl. CPC G09B 5/02; G06F 3/014; G06T 17/00; G06T 2210/41

(57) ABSTRACT

Surgical teaching auxiliary system using virtual reality and method thereof are disclosed. In the Surgical teaching auxiliary system, virtual-reality surgical environment is created through three-dimensional reconstruction based on a two-dimensional surgical image, and an operation step, a time point of using instrument and a reference message are preset in advance; when a surgical training is performed in the virtual-reality surgical environment, a surgical operation behavior is continuously detected; when the surgical operation behavior abnormally interrupted or delayed is detected, an auxiliary support and guidance are triggered based on a progress of the surgical training, thereby achieving the technical effect of greatly improving the effectiveness of surgical learning in the virtual reality.

### CROSS REFERENCE STATEMENT

The present application is based on, and claims priority from, U.S. Provisional Pat. Application Serial Number 63330792, filed Apr. 14, 2022, the disclosure of which is hereby incorporated by reference herein in its entirety.

### BACKGROUND

### 1. Technical Field

The present invention relates to a surgical teaching auxiliary system and a method thereof, more particularly to a surgical teaching auxiliary system using virtual reality and a method thereof.

### 2. Description of the Related Art

In recent years, with the popularization and vigorous development of virtual reality, various applications based on virtual reality have sprung up. In addition to common applications in games, virtual reality is also often applied in teaching, for example, surgery simulation teaching, aviation flight simulation teaching, and other simulation teaching and training.

Generally speaking, the conventional surgical training methods or teaching methods are performed with remains or animals, but this method has the problem of irreversibility and high cost. Therefore, some manufacturers apply virtual reality in surgical teaching, to simulate human organs or demonstrate the surgical process. However, the simulated human organs are still not realistic enough for teaching or training; on the other hand, the degree of freedom of surgical operations in virtual reality is also insufficient. Therefore, the application of virtual reality (VR) in surgical teaching still has limitations.

In view of this, some manufacturers proposed technologies to improve the degree of fidelity and freedom in VR to increase the immersive experience, but high fidelity and freedom do not necessarily improve learning outcomes; for example, when a user does not know what to do next, the operating environment with high fidelity and a high degree of freedom does not assist the user in next operation. Therefore, it is expected to have a positive effect on improving the effectiveness of surgical learning when the system can actively detect whether to provide an auxiliary support and timely intervene in guidance.

According to above-mentioned contents, what is needed is to provide an improved technical solution to solve the conventional problem that the performance of the virtual reality surgery learning is not good enough.

### SUMMARY

An objective of the present invention is to disclose a surgical teaching auxiliary system using virtual reality and a method thereof, to solve the conventional problem that the performance of the virtual reality surgery learning is not good enough.

In order to achieve the objective, the present invention discloses a surgical teaching auxiliary system using virtual reality, and the surgical teaching auxiliary system includes a surgical image database and a host. The surgical image database is configured to store two-dimensional surgical images, wherein each of the two-dimensional surgical images corresponds to a surgical method name, an operation step, a time point of using an instrument, and a reference message. The host is linked to the surgical image database, wherein the host includes a non-transitory computer-readable storage medium and a hardware processor. The non-transitory computer-readable storage medium is configured to store computer-readable program instructions. The hardware processor is electrically connected to the non-transitory computer-readable storage medium, and configured to execute the computer-readable program instructions to execute a creating module, a training module, and a supporting module. The creating module is linked to the surgical image database, wherein before a surgical training is performed, the creating module is configured to receive the surgical method name, load one of the two-dimensional surgical images that corresponds to the received surgical method name, and create virtual-reality surgical environment through 3D reconstruction based on the loaded two-dimensional surgical image. The training module is linked to the creating module, wherein after the virtual-reality surgical environment is created completely, the training module is configured to perform a surgical training, wherein when the surgical training is performed, the training module is configured to continuously detect a surgical operation behavior, and generate a time point message based on a progress of the surgical training when the surgical operation behavior is abnormally interrupted or delayed. The supporting module is linked to the training module, and configured to display the loaded two-dimensional surgical image based on the generated time point message, and synchronously display the operation step, the time point of using instrument, and the reference message, to provide an auxiliary support and guidance.

The present invention discloses a surgical teaching auxiliary method using virtual reality, the surgical teaching auxiliary method is executed by a host linked to a surgical image database, and includes the following steps: storing two-dimensional surgical images in the surgical image database, wherein each of the two-dimensional surgical images corresponds to a surgical method name, an operation step, a time point of using instrument, and a reference message; before a surgical training is performed, receiving a surgical method name to load one of the two-dimensional surgical image that corresponds to the surgical method name, and creating virtual-reality surgical environment through 3D reconstruction based on the loaded two-dimensional surgical image, by the host; after the virtual-reality surgical environment is created completely, permitting to perform the surgical training, continuously detecting a surgical operation behavior when the surgical training is performed, generating a time point message based on a progress of the surgical training when the surgical operation behavior is interrupted or delayed abnormally, by the host; displaying the loaded two-dimensional surgical image, synchronously displaying the operation step, the time point of using instrument, and the reference message for an auxiliary support and guidance based on the generated time point message, by the host.

According to the above-mentioned system and the method of the present invention, the difference between the present invention and the conventional technology is that, in the present invention, the virtual-reality surgical environment is created through the three-dimensional reconstruction based on the two-dimensional surgical image; the operation step, the time point of using instrument and the reference message are preset in advance; when the surgical training is performed in the virtual-reality surgical environment, the surgical operation behavior is continuously detected; when the surgical operation behavior abnormally interrupted or delayed is detected, the auxiliary support and guidance are triggered based on the progress of the surgical training.

Therefore, the above-mentioned technical solution is able to achieve the technical effect of greatly improving the effectiveness of surgical learning in the virtual reality.

### BRIEF DESCRIPTION OF THE DRAWINGS

The structure, operating principle and effects of the present invention will be described in detail by way of various embodiments which are illustrated in the accompanying drawings.

FIG. 1 is a system block diagram of a surgical teaching auxiliary system using virtual reality, according to the present invention.

FIG. 2A and FIG. 2B are flowcharts of a surgical teaching auxiliary method using virtual reality, according to the present invention.

FIG. 3 is a schematic view of a surgery operation in virtual reality, according to an application of the present invention.

FIG. 4 is a schematic view of an operation of detecting a surgical operation behavior interrupted or delayed abnormally, according to an application of the present invention.

### DETAILED DESCRIPTION

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims.

These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the singular forms “a”, “an” and “the” are intended to include the plural forms as well, unless the context clearly indicates otherwise.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the words “comprise” and “include”, and variations such as “comprises”, “comprising”, “includes”, or “including”, will be acknowledged to imply the inclusion of stated elements but not the exclusion of any other elements.

Before illustration for the surgical teaching auxiliary system using virtual reality and the method disclosed in the present invention, the environment where the present invention is applied is described first. The present invention is applied in virtual reality, the virtual reality refers to using computer to simulate and generate a three-dimensional virtual world, so as to provide a user with simulations of vision and other senses, thereby allowing the user to feel as if they are personally in the scene and observe things in three-dimensional space immediately and without limitation.

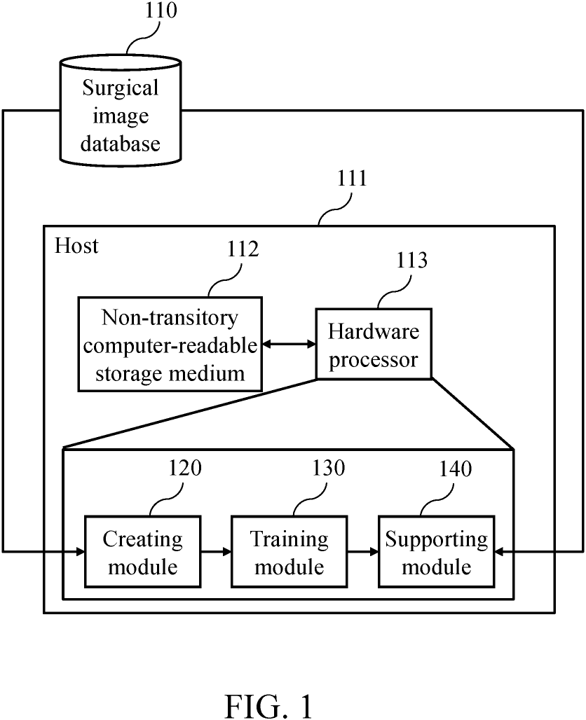

The surgical teaching auxiliary system using virtual reality and method of the present invention will be further described with reference to figures in the following paragraphs. Please refer to FIG. 1, FIG. 1 is a system block diagram of a surgical teaching auxiliary system using virtual reality, according to the present invention. The system includes a surgical image database 110 and a host 111. The surgical image database 110 is configured to store two-dimensional surgical images, each of the two-dimensional surgical images corresponds to a surgical method name, an operation step, a time point of using instrument and a reference message. For example, a two-dimensional surgical image can correspond to a surgical method name being myomectomy, the operation step is at least one execution step of this operation, time points of using instruments record the various surgical instruments needed for this operation, and the time points of using these surgical instruments; the reference message records at least one messages that needs to be paid attention to when performing this operation. In an embodiment, the surgical image database 110 can be independently set outside the host 111 or set directly inside the host 111.

The host 111 is linked to the surgical image database 110, and includes a non-transitory computer-readable storage medium 112 and a hardware processor 113. The non-transitory computer-readable storage medium 112 is configured to store computer-readable program instructions, the hardware processor 113 is electrically connected to the non-transitory computer-readable storage medium 112 and configured to execute the computer-readable program instruction to execute a creating module 120, a training module 130 and a supporting module 140. The creating module 120 is linked to the surgical image database 110; before a surgical training is performed, the creating module 120 receives a surgical method name, and selects and loads one of the two-dimensional surgical images that corresponds to surgical method name; the creating module 120 creates virtual-reality surgical environment through three-dimensional (3D) reconstruction according to the loaded two-dimensional surgical images. In actual implementation, the 3D reconstruction refers to use mathematic process and computer technology of two-dimensional projection or image to recover 3D information (shape, etc.) of an object. The 3D reconstruction can be implemented by optical measure, geometry, deep learning algorithm, to transform the two-dimensional surgical image into 3D information of virtual reality.

The training module 130 is linked to the creating module 120; after the virtual-reality surgical environment is created completely, the training module 130 permits to perform the surgical training; when the surgical training is performed, the training module 130 continuously detects a surgical operation behavior; when the surgical operation behavior is abnormally interrupted or delayed, the training module 130 generates a time point message based on a progress of the surgical training. In actual implementation, the surgical operation behaviors that are not interrupted or delayed abnormally are inputted into the machine learning model for training, and after the training is completed, the detected surgical operation behavior is inputted into the machine learning model, to recognize whether the surgical operation behavior is interrupted or delayed abnormally. In addition, the surgical operation behavior can be detected by at least one of a motion sensor, a wrist-worn device, and a hand motion input device.

The supporting module 140 is linked to the training module 130 and configured to display the loaded two-dimensional surgical images and synchronously display the operation step, the time point of using instrument, and the reference message for an auxiliary support and guidance, based on the generated time point message. For example, in a condition that the generated time point message is ten minutes and thirty seconds, the supporting module 140 starts displaying the loaded two-dimensional surgical image at ten minutes and thirty seconds, and simultaneously displays the corresponding operation step, time point of using instrument and reference message, for an auxiliary support and guidance. In other words, when the use’s surgical operation behavior is interrupted or delayed abnormally, the supporting module 140 immediately displays the surgery operation steps at and after this time point, displays the time point of using the surgery instrument, and provides the user reference message including normal physiological data and notes at this time point.

It is to be particularly noted that, in actual implementation, the modules of the present invention can be implemented by hardware processor, such as integrated circuit chip, system on chip (SOC), a complex programmable logic device (CPLD), or a field programmable gate array (FPGA). The concept of the present invention can be implemented by a system, a method and/or a computer program. The computer program can include computer-readable storage medium which records computer readable program instructions, and the processor can execute the computer readable program instructions to implement concepts of the present invention. The computer-readable storage medium can be a tangible apparatus for holding and storing the instructions executable of an instruction executing apparatus. Computer-readable storage medium can be, but not limited to electronic storage apparatus, magnetic storage apparatus, optical storage apparatus, electromagnetic storage apparatus, semiconductor storage apparatus, or any appropriate combination thereof. More particularly, the computer-readable storage medium can include a hard disk, an RAM memory, a read-only-memory, a flash memory, an optical disk, a floppy disc or any appropriate combination thereof, but this exemplary list is not an exhaustive list. The computer-readable storage medium is not interpreted as the instantaneous signal such a radio wave or other freely propagating electromagnetic wave, or electromagnetic wave propagated through waveguide, or other transmission medium (such as optical signal transmitted through fiber cable), or electric signal transmitted through electric wire. Furthermore, the computer readable program instruction can be downloaded from the computer-readable storage medium to each calculating/processing apparatus, or downloaded through network, such as internet network, local area network, wide area network and/or wireless network, to external computer equipment or external storage apparatus. The network includes copper transmission cable, fiber transmission, wireless transmission, router, firewall, switch, hub and/or gateway. The network card or network interface of each calculating/processing apparatus can receive the computer readable program instructions from network, and forward the computer readable program instruction to store in computer-readable storage medium of each calculating/processing apparatus. The computer program instructions for executing the operation of the present invention can include source code or object code programmed by assembly language instructions, instruction-set-structure instructions, machine instructions, machine-related instructions, micro instructions, firmware instructions or any combination of one or more programming language. The programming language include object oriented programming language, such as Common Lisp, Python, C++, Objective-C, Smalltalk, Delphi, Java, Swift, C#, Perl, Ruby, and PHP, or regular procedural programming language such as C language or similar programming language. The computer readable program instruction can be fully or partially executed in a computer, or executed as independent software, or partially executed in the client-end computer and partially executed in a remote computer, or fully executed in a remote computer or a server.

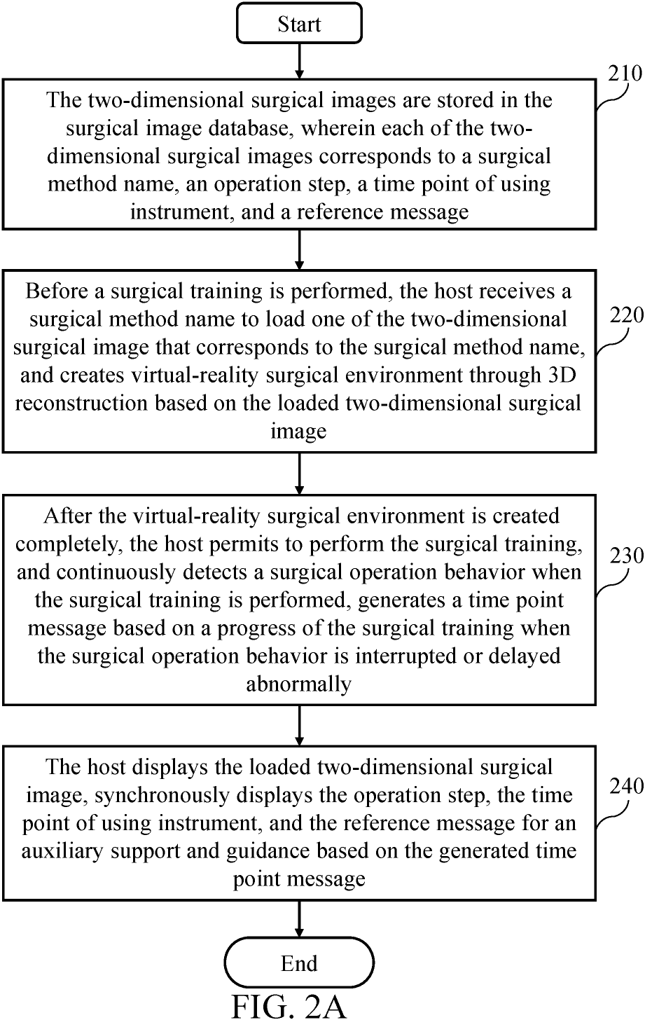

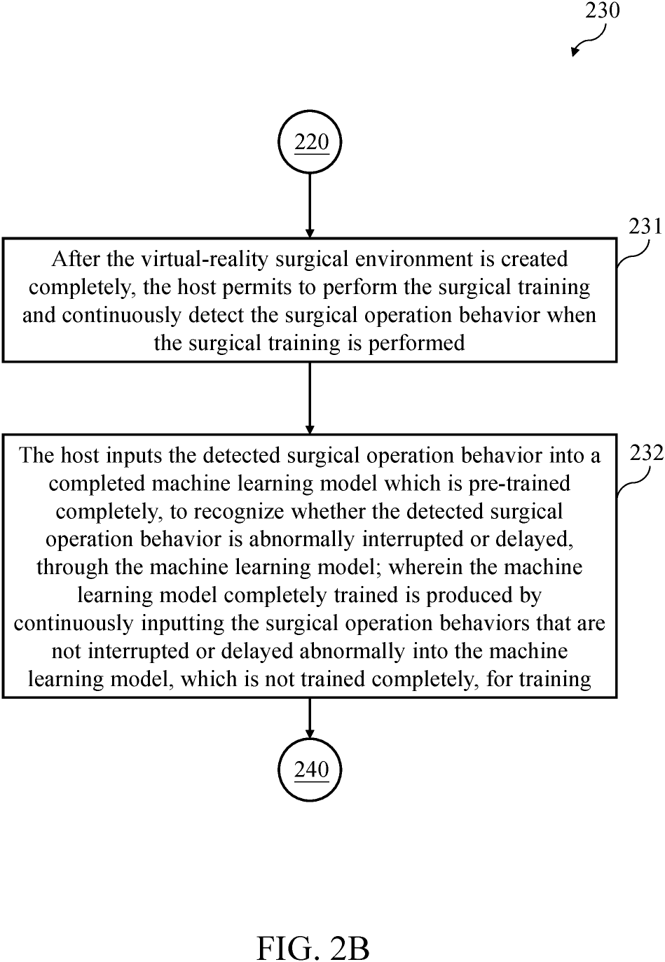

Please refer to FIG. 2A and FIG. 2B, FIG. 2A and FIG. 2B are flowcharts of a surgical teaching auxiliary method using virtual reality, according to the present invention. As shown in FIG. 2A, the surgical teaching auxiliary method is executed by the host 111 linked to the surgical image database 110 and includes the following steps. In a step 210, the two-dimensional surgical images are stored in the surgical image database 110, wherein each of the two-dimensional surgical images corresponds to a surgical method name, an operation step, a time point of using instrument, and a reference message. In a step 220, before a surgical training is performed, the host 111 receives a surgical method name to load one of the two-dimensional surgical image that corresponds to the surgical method name, and creates virtual-reality surgical environment through 3D reconstruction based on the loaded two-dimensional surgical image. In a step 230, after the virtual-reality surgical environment is created completely, the host 111 permits to perform the surgical training, and continuously detects a surgical operation behavior when the surgical training is performed, generates a time point message based on a progress of the surgical training when the surgical operation behavior is interrupted or delayed abnormally. In a step 240, the host 111 displays the loaded two-dimensional surgical image, synchronously displays the operation step, the time point of using instrument, and the reference message for an auxiliary support and guidance based on the generated time point message.

In addition, as shown in FIG. 2B, the step 230 can include two steps. In a step 231, after the virtual-reality surgical environment is created completely, the host 111 permits to perform the surgical training and continuously detect the surgical operation behavior when the surgical training is performed. In a step 232, the host 111 inputs the detected surgical operation behavior into a completed machine learning model which is pre-trained completely, to recognize whether the detected surgical operation behavior is abnormally interrupted or delayed, through the machine learning model. The machine learning model completely trained is produced by continuously inputting the surgical operation behaviors that are not interrupted or delayed abnormally into the machine learning model, which is not trained completely, for training.

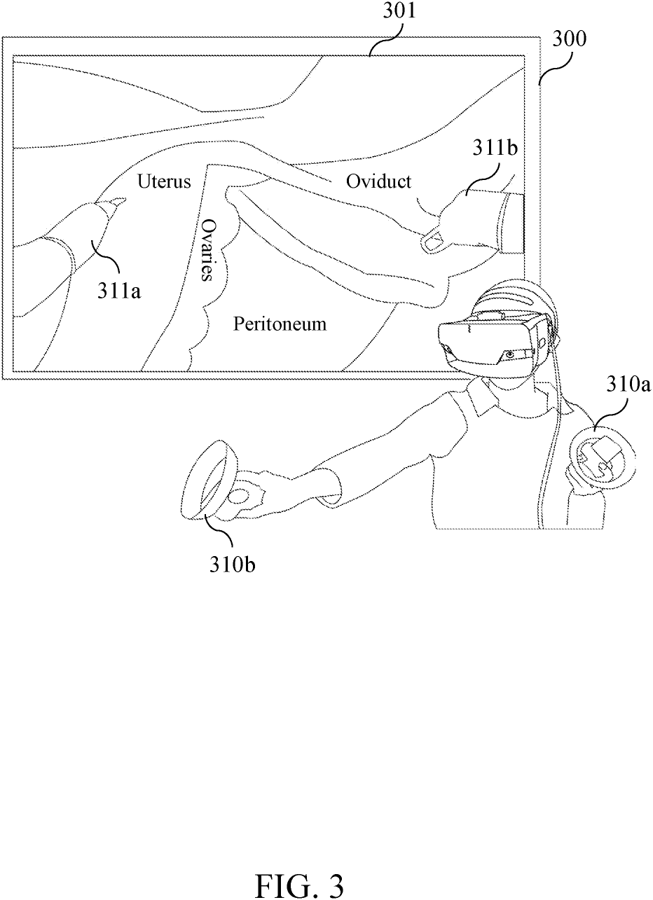

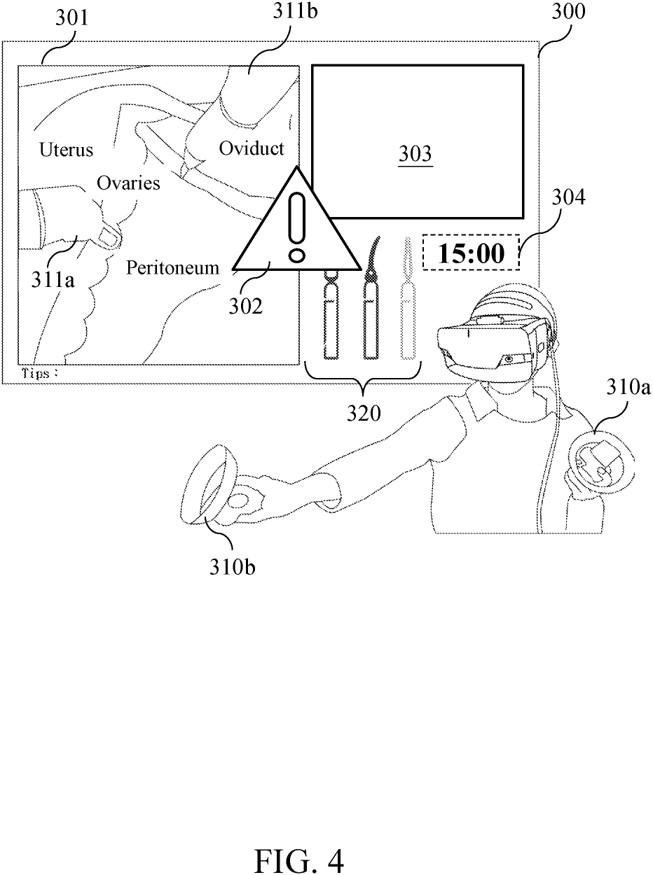

The embodiment of the present invention will be illustrated with reference to FIG. 3 and FIG. 4 in the following paragraphs. FIG. 3 is a schematic view of a surgery operation in virtual reality, according to an application of the present invention. Before a user performs a virtual reality surgery operation, the user inputs a surgical method name (such as myomectomy) through a controller 310b, to load the corresponding two-dimensional surgical image from the surgical image database 110. Next, the creating module 120 loads the loaded two-dimensional surgical image to create virtual-reality surgical environment through 3D reconstruction; when the creation is completed, the user can browse the virtual surgery environment through a first display block 301 on a graphical user interface 300, and the user can control virtual surgical instruments 311a, 311b to perform a surgical training by operating the controller 310a and 310b, respectively. In this case, the training module 130 continuously detects surgical operation behaviors; when a surgical operation behavior is interrupted or delayed abnormally, the training module 130 generates a time point message based on a progress of the surgical training, so that the supporting module 140 plays the loaded two-dimensional surgical image based on the time point message. For example, in a condition that the surgical operation behavior is detected to be interrupted or delayed abnormally in the surgical training at ten minutes and thirty seconds, the time point message is ten minutes and thirty seconds, so that the supporting module 140 starts playing the loaded two-dimensional surgical image from the time point of ten minutes and thirty seconds, and synchronously displaying the operation step, the time point of using instrument, and the reference message, for an auxiliary support and guidance.

Please refer to FIG. 4, FIG. 4 is a schematic view of an operation of detecting a surgical operation behavior interrupted or delayed abnormally, according to an application of the present invention. In actual implementation, when the training module 130 detects that the surgical operation behavior is interrupted or delayed abnormally, as shown in FIG. 4, the training module 130 displays a warning icon 302, and displays the first display block 301, a second display block 303 and a time point display block 304 at the same time. The second display block 303 displays the loaded two-dimensional surgical image based on the time point message, synchronously displays the operation steps, the time points of using instruments, and the reference messages, for an auxiliary support and guidance; the time point display block 304 displays the time point message, for example, the time point of the fifteen minutes can be displayed as 15:00. In actual implementation, the surgical instrument icons 320 to be used can be displayed based on the time point of using instrument in sequential order, so that the user is mentally prepared to switch surgical instruments. In other words, when the training module 130 detects that the surgical operation behavior is interrupted or delayed abnormally, the training module 130 displays the information to support and guide the user, instead of simply displaying the first display block 301 and the virtual surgical environment. For example, the warning icon 302 is used for reminding the user that the current surgical operation behavior is interrupted or delayed abnormally, and the second display block 303 is used for displaying the two-dimensional surgical image corresponding to the time point where the surgical operation behavior is interrupted or delayed abnormally, the time point display block 304 is used for displaying the time point where the surgical operation behavior is interrupted or delayed abnormally, even the surgical instrument icons 320 to be used are displayed in sequential order after this time point. In this way, when the user is not sure about the next step or is completely unclear about the next step, the system of the present invention can immediately intervene in an auxiliary support and guidance, so as to greatly improve the effectiveness of surgical learning in the virtual reality.

According to above-mentioned contents, the difference between the present invention and the conventional technology is that, in the present invention, the virtual-reality surgical environment is created through the three-dimensional reconstruction based on the two-dimensional surgical image; the operation step, the time point of using instrument and the reference message are preset in advance; when the surgical training is performed in the virtual-reality surgical environment, the surgical operation behavior is continuously detected; when the surgical operation behavior abnormally interrupted or delayed is detected, the auxiliary support and guidance are triggered based on the progress of the surgical training. Therefore, the above-mentioned technical solution is able to solve the conventional problem, to achieve the technical effect of greatly improving the effectiveness of surgical learning in the virtual reality.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A surgical teaching auxiliary system using virtual reality, comprising:
a surgical image database, configured to store two-dimensional surgical images, wherein each of the two-dimensional surgical images corresponds to a surgical method name, an operation step, a time point of using an instrument, and a reference message; and
a host, linked to the surgical image database, wherein the host comprises:
a non-transitory computer-readable storage medium, configured to store computer-readable program instructions; and
a hardware processor, electrically connected to the non-transitory computer-readable storage medium, and configured to execute the computer-readable program instructions to execute:
a creating module, linked to the surgical image database, wherein before a surgical training is performed, the creating module is configured to receive the surgical method name, load one of the two-dimensional surgical images that corresponds to the received surgical method name, and create virtual-reality surgical environment through 3D reconstruction based on the loaded two-dimensional surgical image;
a training module, linked to the creating module, wherein after the virtual-reality surgical environment is created completely, the training module is configured to perform a surgical training, wherein when the surgical training is performed, the training module is configured to continuously detect a surgical operation behavior, and generate a time point message based on a progress of the surgical training when the surgical operation behavior is abnormally interrupted or delayed; and
a supporting module, linked to the training module, and configured to display the loaded two-dimensional surgical image based on the generated time point message, and synchronously display the operation step, the time point of using instrument, and the reference message, to provide an auxiliary support and guidance.

2. The surgical teaching auxiliary system using virtual reality according to claim 1, wherein the training module inputs the detected surgical operation behavior into a machine learning model which is trained completely, to recognize whether the detected surgical operation behavior is abnormally interrupted or delayed, through the machine learning model.

3. The surgical teaching auxiliary system using virtual reality according to claim 2, wherein the machine learning model which is trained completely is produced by continuously inputting the surgical operation behavior that has no abnormally interrupted or delayed condition into the machine learning model that has not been trained.

4. The surgical teaching auxiliary system using virtual reality according to claim 1, wherein the three-dimensional reconstruction is performed through at least one of optical measure, geometry, deep learning algorithm, to transform the loaded two-dimensional surgical image into 3D information of virtual reality.

5. The surgical teaching auxiliary system using virtual reality according to claim 1, wherein the surgical operation behavior is detected by at least one of a motion sensor, a wrist-worn device, and a hand motion input device.

6. A surgical teaching auxiliary method using virtual reality, wherein the surgical teaching auxiliary method is executed by a host linked to a surgical image database and comprises:
storing two-dimensional surgical images in the surgical image database, wherein each of the two-dimensional surgical images corresponds to a surgical method name, an operation step, a time point of using instrument, and a reference message;
before a surgical training is performed, receiving a surgical method name to load one of the two-dimensional surgical image that corresponds to the surgical method name, and creating virtual-reality surgical environment through 3D reconstruction based on the loaded two-dimensional surgical image, by the host;
after the virtual-reality surgical environment is created completely, permitting to perform the surgical training, continuously detecting a surgical operation behavior when the surgical training is performed, generating a time point message based on a progress of the surgical training when the surgical operation behavior is interrupted or delayed abnormally, by the host; and
displaying the loaded two-dimensional surgical image, synchronously displaying the operation step, the time point of using instrument, and the reference message for auxiliary support and guidance based on the generated time point message, by the host.

7. The surgical teaching auxiliary method using virtual reality according to claim 6, further comprising:
inputting the detected surgical operation behavior into a machine learning model which is trained completely, to recognize whether the detected surgical operation behavior is abnormally interrupted or delayed, through the machine learning model, by the training module.

8. The surgical teaching auxiliary method using virtual reality according to claim 7, wherein the machine learning model which is trained completely is produced by continuously inputting the surgical operation behavior that has no abnormally interrupted or delayed condition into the machine learning model that has not been trained.

9. The surgical teaching auxiliary method using virtual reality according to claim 6, wherein the three-dimensional reconstruction is performed through at least one of optical measure, geometry, deep learning algorithm, to transform the loaded two-dimensional surgical image into 3D information of virtual reality.

10. The surgical teaching auxiliary method using virtual reality according to claim 6, wherein the surgical operation behavior is detected by at least one of a motion sensor, a wrist-worn device, and a hand motion input device.
